(19) United States
(12) Patent Application Publication
(10) Pub. No.: US 2026/0249106 A1
(43) Pub. Date: Aug. 27, 2026

(54) Devices, Systems and Methods for Low Intensity Volumetric Ultrasound (LIVU) Treatment of Subcutaneous Tissue

(71) Applicant: BiLLite Medical LLC, Shreveport, LA (US)

(72) Inventors: Gerard Dayle Henry, Shreveport, LA (US); Robert Allen Van Wyk, St. Petersburg, FL (US); Raymond Mocco, Easton, CT (US)

(21) Appl. No.: 19/546,404

(22) Filed: Feb. 22, 2026

Related U.S. Application Data
(60) Provisional application No. 63/762,597, filed on Feb. 24, 2025.

(51) Int. Cl. A61N 7/00

(52) U.S. Cl. CPC A61N 7/00; A61N 2007/0008; A61N 2007/0047; A61N 2007/0052

(57) ABSTRACT

Devices, systems, and methods for treatment of the subcutaneous tissues of various types. Non-invasive ultrasound energy is used for planning, monitoring and treating the adipose tissue, small blood vessels, and benign tumors of the subcutaneous tissue. While high intensity focal ultrasound (HIFU) has been used to treat many areas of the body including cancer lesions typically using ablation/high heat energy, lower intensity volumetric ultrasound (LIVU) techniques are used to denature the subcutaneous layer. Various safety features may be included to further enhance the treatment of the subcutaneous tissue using LIVU techniques.

### RELATED APPLICATIONS

The present application claims priority to U.S. Provisional Application No. 63/762,597, filed on Feb. 24, 2025.

### TECHNICAL FIELD

Embodiments disclosed herein relate generally to the field of ultrasound, and more particularly, to ultrasound surgery devices and methods to treat the subcutaneous layer.

### BACKGROUND

Currently, for medical issues of the subcutaneous tissue, treatments are either external to the skin and are mostly weak and ineffective or the other extreme that may require surgical excision with significant anesthesia requirements. Medical issues of the subcutaneous layer have been limited to surgical excision (including the excision of a lipoma, open fat removal, or liposuction) or application of transdermal pads (“coolsculpting”) which can be weak and ineffective for treating this layer of the body.

Surgical excision and liposuction treatments usually require systemic anesthesia (MAC or General) with the inherent and associated risks. This is in addition to the morbidity of surgical excision including bleeding, infection, injury to nearby tissue, pain issues, scarring, and poor healing. As stated previously, external treatments are largely ineffective for treating subcutaneous tissue conditions as they must have energy transfer directly through the skin.

There is currently a need for a device that delivers a defined amount of energy to the subcutaneous layer that avoids dermal layer damage while treating the subcutaneous tissue. The treatment needs to be provided to ensure that both adequate treatment goals are achieved, and safety parameters are maintained. For instance, treating a lipoma (benign fat tumor) currently requires open surgical removal, rather than transdermal treatment methods, which are often suboptimal treatment options. Thus, there is a need for improved devices and methods for the treatment of subcutaneous tissue with reduced risk factors. Such devices and methods are the subject of various embodiments described herein.

Basic Skin Anatomy: Referring to FIG. 1, skin 900 is composed of 3 layers: the epidermis 902 is the outermost layer and is composed of keratinocytes or skin cells that form the “bricks” of the skin's barrier. The functions of the epidermis are protection from environmental insults (like ultraviolet light and toxins), prevention of dryness, and immune surveillance. The base of the epidermis is called the basal layer 904—it contains the cells that replicate to replace the epidermis every month. Beneath the epidermis is the dermis 906, which is composed mostly of collagen but also adjunctive structures like hair follicles and sweat glands. The dermis also contains vital blood vessels and nerves which traverse the collagen network there. The function of the dermis is temperature regulation though the secretion of sweat to the skin's surface and the regulation of blood flow to the area. Below the dermis, lies the subcutis [or hypodermis] 908 which holds fat and blood vessels. Fat is arranged into lobules that are several millimeters wide. The subcutis 908 acts as a heat insulator and provides protection from mechanical force or impact induced trauma.

Currently, technology for high intensity focal ultrasound (HIFU) treatment uses tissue ablation with large amounts of energy to treat tissue. HIFU high energy treatments are used to ablate several cancers inside the body, mainly through the skin with no damage to the skin. For instance, HIFU is used for the treatment of prostate cancer through the rectal wall. When used for prostate cancer treatment, the HIFU generator, using ultrasound imaging, will not treat within 5 mm of the rectum, and the HIFU generator, using ultrasound imaging, ablates the treated tissue with such high heat that the tissue can be seen “melting” in real-time.

### SUMMARY

Embodiments disclosed herein address issues such as these and others by providing low intensity volumetric ultrasound (LIVU) systems and methods that include aspects that provide for increased efficacy and/or safety when treating the subcutaneous tissue. For instance, at least some embodiments may provide LIVU treatment configured to only treat until a treatment endpoint (for example a predetermined acoustic change to avoid dangerous necrotic tissue build up) is reached, such as where denaturation of the subcutaneous tissue has occurred. At least some embodiments may provide for monitoring the acoustic impedance during delivery of LIVU energy to the tissue to ensure safety and efficacy of the therapy. At least some embodiments may be configured to utilize an ultrasound analysis to develop a treatment plan for the LIVU therapy. At least some embodiments may be configured to perform real-time imaging during the LIVU therapy. At least some embodiments may monitor skin temperature during the LIVU therapy. Each of the acoustic impedance monitoring, ultrasound imaging, and/or temperature monitoring aspects may be used as treatment endpoints to terminate the LIVU therapy to ensure safety and efficacy. At least some embodiments may utilize a template to mark the skin where the LIVU therapy is to be applied. At least some embodiments may provide LIVU therapy using a phased array transducer. At least some embodiments may utilize power adjustments to set the depth of the LIVU therapy. At least some embodiments may utilize pulse trains to provide the LIVU therapy. At least some embodiments may utilize a wave emitter pattern to set a depth of the LIVU therapy. At least some embodiments may use a local anesthesia for the LIVU therapy. At least some embodiments may utilize a power density threshold when providing LIVU therapy. At least some embodiments may monitor a physiological parameter and provide a remedial action if necessary when providing LIVU therapy.

Embodiments provide a method of treating subcutaneous tissue that involves applying low intensity volumetric ultrasound to the subcutaneous tissue at an area to be treated. The method further involves continuing the application of the low intensity volumetric ultrasound until the subcutaneous tissue at the area to be treated has achieved a treatment endpoint.

Embodiments provide a method of treating subcutaneous tissue that involves performing an ultrasound analysis at a subcutaneous depth over an area to be treated to develop a treatment plan. The method further involves applying low intensity volumetric ultrasound into the subcutaneous tissue at the area to be treated according to the treatment plan.

Embodiments provide a method of treating subcutaneous tissue that involves applying low intensity volumetric ultrasound into subcutaneous tissue at an area to be treated. The method further involves monitoring acoustic impedance to determine when the subcutaneous tissue at the area to be treated has met a treatment endpoint.

Embodiments provide a method of treating subcutaneous tissue that involves applying low intensity volumetric ultrasound into subcutaneous tissue at an area to be treated. The method further involves performing real-time imaging to monitor the subcutaneous tissue to be treated to determine when the subcutaneous tissue at the area to be treated has achieved a treatment endpoint.

Embodiments provide a method of treating subcutaneous tissue that involves applying low intensity volumetric ultrasound into subcutaneous tissue at an area to be treated. The method further involves monitoring the temperature of skin of a patient over the area containing the subcutaneous tissue to be treated to determine when the subcutaneous tissue at the area to be treated has met a treatment endpoint.

Embodiments provide a method of treating subcutaneous tissue that involves utilizing a template to mark skin of a patient over an area containing the subcutaneous tissue to be treated. The method further involves applying low intensity volumetric ultrasound into the subcutaneous tissue at the area to be treated until the subcutaneous tissue at the area achieves a treatment endpoint.

Embodiments provide a method of treating subcutaneous tissue that involves applying low intensity volumetric ultrasound into the subcutaneous tissue at an area to be treated using phased array ultrasonic transducer with an ability to modify focusing characteristics during use so as to create a predetermined treatment volume. The method further involves continuing the application of the low intensity volumetric ultrasound into the subcutaneous tissue at the area to be treated until the subcutaneous tissue at the area achieves a treatment endpoint.

Embodiments provide a method of treating subcutaneous tissue that involves adjusting power of a low intensity volumetric ultrasound device to set a depth of treatment to be that of subcutaneous tissue at an area to be treated. The method further involves applying, by the low intensity volumetric ultrasound device with the adjusted power, low intensity volumetric ultrasound into the subcutaneous tissue at the area to be treated until the subcutaneous tissue at the area to be treated has achieved a treatment endpoint.

Embodiments provide a method of treating subcutaneous tissue that involves applying low intensity volumetric ultrasound as pulse trains into subcutaneous tissue at an area to be treated. The method further involves continuing the application of the pulse trains until the subcutaneous tissue at the area to be treated has achieved a treatment endpoint.

Embodiments provide a method of treating subcutaneous tissue that involves adjusting a wave emitter pattern of a low intensity volumetric ultrasound device to set a depth of treatment to be that of subcutaneous tissue at an area to be treated. The method further involves applying, by the low intensity volumetric ultrasound device with the adjusted wave emitter pattern, low intensity volumetric ultrasound into the subcutaneous tissue at the area to be treated until the subcutaneous tissue at the area to be treated has achieved a treatment endpoint.

Embodiments provide a method of treating subcutaneous tissue that involves applying a local anesthesia to skin of the patient over an area to be treated. The method further involves applying low intensity volumetric ultrasound through the skin where the local anesthesia is applied and into the subcutaneous tissue at the area to be treated until the subcutaneous tissue at the area to be treated has achieved a treatment endpoint.

Embodiments provide a method of treating subcutaneous tissue that involves applying low intensity volumetric ultrasound with a power density level not exceeding. The method further involves continuing the application of the low intensity volumetric ultrasound until the subcutaneous tissue at the area to be treated has achieved a treatment endpoint.

Embodiments provide a method of treating subcutaneous tissue that involves applying low intensity volumetric ultrasound to subcutaneous tissue at an area to be treated. The method further involves monitoring a physiological parameter while applying the low intensity volumetric ultrasound. The method also involves, upon detecting that the physiological parameter meets a threshold, taking a remedial action.

Embodiments provide a system that treats subcutaneous tissue that includes an ultrasound device configured to apply low intensity volumetric ultrasound to the subcutaneous tissue at an area to be treated. The ultrasound device continues the application of the low intensity volumetric ultrasound until the subcutaneous tissue at the area to be treated has achieved a treatment endpoint.

Embodiments provide a system for treating subcutaneous tissue that includes an ultrasound device configured to perform an ultrasound analysis at a subcutaneous depth over an area to be treated to develop a treatment plan. The ultrasound device is further configured to apply low intensity volumetric ultrasound into the subcutaneous tissue at the area to be treated according to the treatment plan.

Embodiments provide a system for treating subcutaneous tissue that includes an ultrasound device configured to apply low intensity volumetric ultrasound into subcutaneous tissue at an area to be treated. The ultrasound device is further configured to monitor acoustic impedance to determine when the subcutaneous tissue at the area to be treated has met a treatment endpoint.

Embodiments provide a system for treating subcutaneous tissue that includes an ultrasound device configured to apply low intensity volumetric ultrasound into subcutaneous tissue at an area to be treated. The ultrasound device is further configured to perform real-time imaging to monitor the subcutaneous tissue to be treated to determine when the subcutaneous tissue at the area to be treated has achieved a treatment endpoint.

Embodiments provide a system for treating subcutaneous tissue that includes an ultrasound device configured to apply low intensity volumetric ultrasound into subcutaneous tissue at an area to be treated. The ultrasound device is further configured to monitor the temperature of skin of a patient over the area containing the subcutaneous tissue to be treated to determine when the subcutaneous tissue at the area to be treated has met a treatment endpoint.

Embodiments provide a system for treating subcutaneous tissue that includes a template to guide marking skin of a patient over an area containing the subcutaneous tissue to be treated. The system further comprises an ultrasound device configured to apply low intensity volumetric ultrasound into the subcutaneous tissue at the area to be treated based on marks on the skin until the subcutaneous tissue at the area achieves a treatment endpoint.

Embodiments provide a system for treating subcutaneous tissue that includes an ultrasound device configured to apply low intensity volumetric ultrasound into the subcutaneous tissue at an area to be treated using a phased array ultrasonic transducer with an ability to modify focusing characteristics during use so as to create a predetermined treatment volume. The ultrasound device is further configured to continue the application of the low intensity volumetric ultrasound into the subcutaneous tissue at the area to be treated until the subcutaneous tissue at the area achieves a treatment endpoint.

Embodiments provide a system for treating subcutaneous tissue that includes an ultrasound device configured to adjust power of a low intensity volumetric ultrasound device to set a depth of treatment to be that of subcutaneous tissue at an area to be treated. The ultrasound device is further configured to apply low intensity volumetric ultrasound with the adjusted power into the subcutaneous tissue at the area to be treated until the subcutaneous tissue at the area to be treated has achieved a treatment endpoint.

Embodiments provide a system for treating subcutaneous tissue that includes an ultrasound device configured to apply low intensity volumetric ultrasound as pulse trains into subcutaneous tissue at an area to be treated. The ultrasound device is further configured to continue the application of the pulse trains until the subcutaneous tissue at the area to be treated has achieved a treatment endpoint.

Embodiments provide a system for treating subcutaneous tissue that includes an ultrasound device configured to adjust a wave emitter pattern of the ultrasound device to set a depth of treatment to be that of subcutaneous tissue at an area to be treated. The ultrasound device is further configured to apply with the adjusted wave emitter pattern a low intensity volumetric ultrasound into the subcutaneous tissue at the area to be treated until the subcutaneous tissue at the area to be treated has achieved a treatment endpoint.

Embodiments provide a system for treating subcutaneous tissue, wherein a local anesthesia is applied to skin of a patient over an area to be treated. The system includes an ultrasound device configured to apply low intensity volumetric ultrasound through the skin where the local anesthesia is applied and into the subcutaneous tissue at the area to be treated until the subcutaneous tissue at the area to be treated has achieved a treatment endpoint.

Embodiments provide a system for treating subcutaneous tissue that includes an ultrasound device configured to apply low intensity volumetric ultrasound with a power density level at or below 50 W/cm². The ultrasound device is further configured to continue the application of the low intensity volumetric ultrasound until the subcutaneous tissue at the area to be treated has achieved a treatment endpoint.

Embodiments provide a system for treating subcutaneous tissue that includes an ultrasound device configured to apply low intensity volumetric ultrasound to subcutaneous tissue at an area to be treated. The ultrasound device is further configured to monitor a physiological parameter while applying the low intensity volumetric ultrasound and upon detecting that the physiological parameter meets a threshold take a remedial action.

### BRIEF DESCRIPTION OF THE FIGURES

FIG. 1 is a diagrammatic depiction of skin anatomy and structures.

FIG. 2 is a plan view of elements of an assembly of an illustrative embodiment for subcutaneous treatment of adipose tissue.

FIG. 3 is a side elevational view of the objects of FIG. 2.

FIG. 4 is a perspective view of the objects of FIG. 2.

FIG. 5 is a sectional view of the objects of FIG. 2 at location A-A.

FIG. 6 is a sectional view of the objects of FIG. 5 wherein the field of focused ultrasonic energy is depicted.

FIG. 7 is a sectional view of the objects of FIG. 5 treating fat lobules according to methods of the various embodiments.

FIG. 8 is a plan view of a template of the illustrative embodiment.

FIG. 9 is a side elevational view of the objects of FIG. 8.

FIG. 10 is a perspective view of the object of FIG. 8.

FIG. 11 is a plan view depiction of subcutaneous tissue to be treated using devices, systems and methods of the various embodiments.

FIG. 12 depicts the template of FIG. 8 in a first position relative to the tissue of FIG. 11, and markings placed by a clinician, the marks providing references for initial positioning of a device of an illustrative embodiment at the start of treatment wherein treatment is accomplished in a single pass.

FIG. 13 depicts the template of FIG. 8 in a second position relative to the tissue of FIG. 11, and markings placed by a clinician, the marks providing references for final positioning of a device of the illustrative embodiment at the completion of treatment.

FIG. 14 depicts markings formed in FIGS. 12 and 13, and a treatment region resulting from use of the device in the region bounded by the clinician markings.

FIG. 15 depicts markings formed by a clinician using the template of FIG. 8 to define a region for treatment of subcutaneous tissue using multiple linear passes of the device over the tissue.

FIG. 16 depicts the tissue and bounding markings of FIG. 15 with the resulting treatment region.

FIG. 17 is a side elevational view of a transducer assembly of the illustrative embodiment.

FIG. 18 is a bottom plan view of the objects of FIG. 17.

FIG. 19 is a perspective view of the objects of FIG. 17.

FIG. 20A is a top plan view of the objects of FIG. 17.

FIG. 20B is a side elevational sectional view of the objects of FIG. 20 at location A-A.

FIG. 21A depicts a path for the beam in the focal plane during use.

FIG. 21B depicts the path of FIG. 21A with the diameter of the beam in the focal plane overlayed.

FIG. 21C depicts the central portion of a focal plane and a coordinate system imposed thereon.

FIG. 22 is a plan view of a handpiece of the illustrative embodiment.

FIG. 23 is a side elevational view of the objects of FIG. 22.

FIG. 24 is a perspective view of the objects of FIG. 22.

FIG. 25 is a is a side elevational view of the objects of FIG. 22 depicting internal elements.

FIG. 26 is a plan view of the handpiece of FIG. 22.

FIG. 27 is a side elevational sectional view of the objects of FIG. 22 at location A-A.

FIG. 28 is a perspective view of a console of the illustrative embodiment, the console providing power to the handpiece of FIG. 22 and receiving signals therefrom.

FIG. 29 is a front elevational view of the objects of FIG. 28.

FIG. 30 depicts a system of the illustrative embodiment for treating subcutaneous tissue using focused ultrasound.

FIG. 31 depicts a pulse train output of an ultrasound transducer.

FIG. 32 depicts the planning and treatment algorithm for LIVU in the denaturing of subcutaneous tissue.

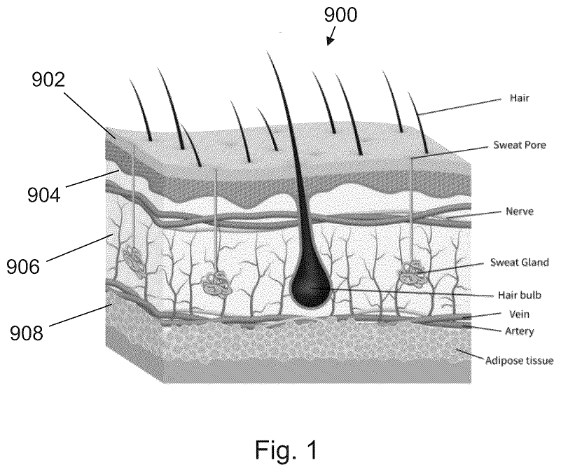

### DETAILED DESCRIPTION

Embodiments disclosed herein provide LIVU techniques for treating subcutaneous tissue while avoiding the major concerns of performing cutaneous HIFU ablation such as thermal injury, pain, and wound healing. Embodiments including methods and devices of LIVU treatment may not treat within a specified distance, such as 10 mm, of the epidermis.

The methods and devices of the various embodiments may use lower energy than HIFU to reach a treatment endpoint where there is just denaturation of the subcutis tissue while also monitoring the treatment. In addition, emitter heads (delivers the energy ultrasound wave for treatment) of the system may be set so that they cannot deliver energy within a specified distance, such as 1 cm, of the end of the LIVU probe. Therefore, methods and devices of the various embodiments will affect tissue below the epidermis and dermis in the subcutaneous layer without thermally affecting the epidermis and dermis layers.

Temperature sensor(s) will be distally facing on the probe to ensure contact with the epidermis before energy is given and to monitor skin temperature during treatment with an alarm energy shut off and/or other remedial action. LIVU energy concentrates at least a specified distance, such as at least 10 mm, below the epidermis and will be applied after ultrasound (US) imaging of the area is done to ensure adequate subcutaneous depth and to ensure no large blood vessels or fluid collections are in the treatment area. In addition, this US imaging may be used to plan treatment. A marking template may be used to ensure that the patient and health care provider agree on the area to be treated.

Methods and devices of the various embodiments affect tissue below the epidermis and dermis in the subcutaneous layer without thermally affecting the epidermis and dermis. LIVU energy is applied in the subcutis to thermally treat larger masses of adipose tissue, benign tumors, and smaller vessels thereby avoiding the epidermis/dermis complex where thermal injury typically occurs that can result in poor wound healing and pain. For example, lipomas [benign tumor made of fat tissue that grows under the skin] currently require open surgical excision. LIVU may be given to the lipoma until changes in the imaging reveal real time denaturing of the tissue.

Embodiments of devices may incorporate multiple ultra-sound emitting heads that pass ultrasound energy through the skin from different points and combine below the dermis to concentrate this energy to the target tissue. As the emitting ultrasound heads are separated at the skin, minimal energy is absorbed by the skin. As a result, thermal effects are limited to regions well below the skin and undesirable dermal effects are avoided. The LIVU ultrasound emitters of various embodiments disclosed herein are positioned apart from each other, and the concentration of energy portions are set to be positioned more than 1 cm below the skin surface before the energy is applied to the subcutaneous substance-also known as hypodermis. Additionally, in existing thermal treatment systems, the algorithm of the HIFU emitting system can be set to not treat tissue depth of less than 5 mm. For the LIVU algorithm of the various embodiments, the minimum depth may be set at a larger depth such as 10 mm. The LIVU probe will have a skin temperature shut off alarm or other remedial action enabling the monitoring of the skin temperature so that the energy level/focusing is kept less than that required to produce dermal injury, but sufficient to treat the region of interest. In at least some embodiments, the hand piece incorporates thermal sensor(s) that monitor skin temperature so as to enable the system to shut off energy if skin temperature rises. The thermal sensor(s) may be located in a distal-facing surface that is pressed against the skin during treatment thereby ensuring that the ultrasound emitter elements probe is fully in contact with the epidermis, thereby preventing superficial activation that could result in dermal injury. In methods of thermal treatment according to various embodiments, ultrasound imaging is used to examine skin and subsurface tissue to ensure that no significant large vessels, fluid collections, or any other abnormalities are present subcutaneously. This ultrasound examination ensures adequate subcutaneous fat depth is present in the area to be treated.

In various embodiments, thermal treatment LIVU systems operate at low energy levels that are much lower than current HIFU treatment regimens. Moreover, instead of Focal Ultrasound, LIVU will use Volumetric Ultrasound to treat a larger area. Typically, when using Focal Ultrasound, only 1 to 2 mm³ of tissue is targeted, whereas Volumetric Ultrasound of various embodiments herein targets 4 or more mm³ of tissue which reduces the likelihood of dangerous concentrated energy delivery which could cause a dangerous increase in the temperature of the treated tissues including the skin. In some systems according to various embodiments, wherein the generator monitors acoustic impedance of the tissue, the generator may use algorithms to detect the completion of treatment by changes in the tissue appearance and alert the clinician or terminate activation. For example, as the ultrasound frequency of the target changes significantly an alert can be indicated by the device and an acoustic impedance alarm can be used to shut off treatment and/or provide other remedial actions.

While HIFU is commonly used for cutting, coagulating, and ablating tissue, the various embodiments of the LIVU methods, devices, and systems are used to reach a treatment endpoint such as to denature the subcutaneous tissue so that the body naturally absorbs the denatured tissue. This is particularly advantageous as it provides clinicians with the ability to concentrate on specific areas that patients have been unable to lose/reduce with standard weight loss management or other body contouring methods. Surgical excision of these small, localized fat deposits results in risks outweighing benefits, for reasons stated already.

When using devices and methods of the various embodiments, only local anesthesia is required. This eliminates the risks and costs associated with systemic general anesthesia.

By avoiding the concentrating flow of HIFU energy to the epidermis and dermal layers of the skin, current devices prevent thermal damage to the skin. Because of this and the non-invasive nature of methods of the present technology, injury to the skin is minimal and patient pain is reduced. With LIVU, the energy only impacts tissue where it combines to concentrate from different emitters. As a result of avoiding the application of HIFU energy to the epidermis and dermis layers of the skin, the patient experiences less direct heating of these tissues resulting in less thermal injury and better post treatment healing of the area.

Systems and methods of current technology for subcutaneous thermal tissue treatment incorporate additional devices that may be supplied to the clinician as a kit. These include a template for marking the location(s) for the treatment. In contrast to treating a patient using methods of the current technologies in the market, the region to be treated is first examined using ultrasound imaging to identify locations for LIVU treatment. Thereafter a template can be used to mark the treatment location(s) using a suitable skin marker. In addition, the patient will be able to see exactly where the treatment is to be given prior to proceeding ahead. The location template device is removed, and the probe of the treatment device is fully engaged until a distal face of the hand piece is pressed against the skin so that a thermal sensor is in firm contact with the skin. The power level for treatment may be selected by the clinician or the LIVU generator may establish it automatically based on safety to the measured depth level and algorithms within the generator on imaging. Thereafter, the generator is activated, and LIVU energy is applied to the target site in short bursts to a predetermined safety value whereupon activation is terminated. The probe is then withdrawn from the site. During activation, the skin temperature is monitored via one or more thermal sensors on the device that are connected by wires to the energy generator. If the skin temperature exceeds a preset safety value during treatment, the supply of LIVU energy to the handpiece is suspended until the temperature falls to an acceptable preset value.

Determining when a given thermal effect has been achieved for a given tissue mass is relatively straightforward. When thermally treating tissue with LIVU devices with the ultrasound emitters mounted at a distance from one another, the energy flow is strongly affected by the location of the treatment depth. Skin and subcutaneous tissue proximal to the treatment site through which the LIVU waves are emitting are protected by the lower energy/power density in the region between the focus plane and the emitter.

Accordingly, when thermally treating tissue using current in-market technology, the tissue type, spacing, and treatment depth are unknown prior to treatment. In some embodiments these variables are supplied to the LIVU generator wherein optimal characteristics for the LIVU output are calculated using algorithms using live ultrasound imaging. In some embodiments, the output of the generator is formed of pulses of LIVU energy. The “on time”, “off time”, and amplitude (power level) of this pulse train may be optimized for thermal treatment of the specified tissue in a manner that allows complete treatment without damage to surrounding tissue. In some embodiments, the ultrasound generator monitors the treatment sites and determines when treatment is complete. The live ultrasound imaging may monitor acoustic changes in the targeted treatment areas.

The ultrasound (US) unit that is typically used to measure ultrasound frequency is hertz (Hz), which represents the number of cycles per second. Ultrasound frequencies are often expressed in megahertz (MHz), where 1 MHz is equal to 1 million hertz. For imaging muscle tissue, frequencies typically range from 7 to 15 MHz, offering detailed visualization of superficial muscles and pathology. Bone imaging poses challenges due to its high acoustic impedance, often necessitating lower frequencies between 0.5 and 2 MHz. Fat imaging encompasses a broad range of frequencies, with 5 to 12 MHz commonly used for subcutaneous fat layers. Higher frequencies (e.g., 15-20 MHz) are often used for imaging superficial structures and for achieving high-resolution images of the epidermis and upper dermis. When ultrasound is used to assess fluid near the skin, there is very low acoustic impedance. The image is usually very dark to black with very high frequencies (over 15 MHz). Ultrasound devices of the various embodiments have a measurement scale limiting treatment to frequencies to a particular range such as from 5 to 12 MHz and limiting treatment energy densities to tissues to a particular range of depths, such as from 1 cm below the skin to 4 cm max depth.

When ultrasound waves travel through tissue, their wavelength and amplitude changes depending on the acoustic impedance of that tissue. As the tissue temperature increases, the ultrasound waves would be absorbed more readily by surrounding tissues leading to decrease in penetration. During HIFU for prostate cancer (during Focal One therapy for example), real-time ultrasound imaging is utilized to monitor the procedure. A notable observation during this process is the lightening or brightening of the prostate tissue on ultrasound imaging as heat is applied. This phenomenon occurs due to several factors. First, the targeted delivery of heat energy to the tissue results in tissue heating, causing changes in its physical properties to reach a treatment endpoint. Additionally, the application of heat leads to the denaturation of proteins within the tissue, altering its acoustic properties and increasing ultrasound reflectivity. Heat-induced vaporization of fluids within the prostate and subsequent formation of gas pockets further contribute to the observed brightening effect on ultrasound images. Lastly, thermal expansion of the tissue also occurs under heat, leading to changes in tissue density and acoustic impedance, which further enhance brightness on ultrasound scans. The US tool, in some embodiments of the LIVU methods, devices, and systems, provides a clinical alert and/or acoustic impedance alarm that stops the energy (heat) delivery when the subcutaneous tissue leads to the denaturation of the tissue causes changes in the wavelength and before the subsequent formation of gas pockets.

Various embodiments of LIVU devices operate at a lower energy/intensity and cover a larger volume of targeted tissue than HIFU to decrease the potential for thermal injury and charring. In some embodiments, an ultrasound image acoustic alarm or other remedial action is set to a level at which the subcutaneous tissue is only denatured to establish a treatment endpoint, and liquification and/or boiling is precluded. The “on time”, “off time”, and amplitude (power level) of the LIVU pulse train may be optimized for thermal treatment of the specified tissue in a manner that allows adequate treatment without injury to surrounding tissue. Ultrasound imaging is used to ensure there is an adequate depth of the subcutaneous tissue prior to treatment and to evaluate the tissue being treated. In some methods of the current technologies, a marking of the skin is used to ensure desired location of treatment to the clinician and the patient. In some embodiments of devices of the current technologies, a temperature probe is used to ensure the distal surface of the device handle is against the skin during treatment and functions as a safety energy shutoff if the skin temperature rises above a predetermined value. The LIVU tool, in some embodiments, has a MHz measurement scale where it will only treat 5 to 12 MHz and can only concentrate energy from some minimum, such as 1 cm below the skin to some maximum, such as a 4 cm max depth. The LIVU generator, in some embodiments, will monitor ultrasound acoustic impedance of the tissue being treated and have an alarm that shuts off energy and/or provides other remedial actions once tissue acoustic impedance is met; therefore, energy would be run though the subcutis tissue and automatically shuts off energy when tissue is treated to be denatured only.

The effect of HIFU energy applied to a tissue is strongly affected by its ultrasound wavelength and thermal conductivity. Ultrasound wave frequency in Hz and thermal conductivity in W/(m·k) for tissue types are given below.

| | | Thermal - |
|---|---|---|
| | | W/(m · k) (watts |
| | US wave range - | per meter per |
| Tissues | Frequency in units of Hz | kelvin) at 39 C. |
| Blood | Drug delivery and blood-brain | .52 |
| | barrier disruption: 0.5-1.5 | |
| | MHz | |
| | Clot distribution and blood | |
| | flow restoration: 1-3 MHz | |
| Blood | Vessel Occlusion and Rupture | .46 |
| vessel wall | Prediction: around 3.3 MHz | |
| | Thermal Effects and | |
| | Coagulation: 1.5-3.3 MHz | |
| Extracellular | Modulation of Extracellular | .60 |
| fluid | Matrix: 1-3 MHz | |
| Fat | Thermal ablation: 1-2 MHz | .21 |
| | Mechanical Disruption: | |
| | 0.8-1.5 MHz | |
| | Enhanced Lipolysis: 1-3 MHz | |
| Muscle | Tumor ablation: 1.5-2.5 | .49 |
| | Musculoskeletal Treatment: 1-2 | |
| | Muscle Regeneration: 1.5-3 | |
| Skin | Skin treatments: 4-7 MHz | .37 |

Blood is very conductive to US, both wave and thermally. Blood vessel walls are less conductive than blood itself but still have fairly high thermal conductivity. This contrasts with fat which has low US wave conductivity and very low thermal conductivity. The power output of a US wave generator is determined by the circuitry within the generator. In systems of the various embodiments, the distance between the US emitters and treatment area of the US emitters is established by a hand piece that may be formed in a manner described herein. With these values fixed, the generator is able to determine the conductivity of the tissue and select a mode optimal for the tissue to be treated. The load curves/modes for treating highly conductive tissues like blood vessels will be very different from those for treating, for instance, fat with its lower US wave and thermal conductivity, even though the treatment of both falls within the scope of this disclosure. Ultrasound imaging is used prior to treatment to evaluate the tissue being treated to plan accordingly. Thereafter, based on information garnered through the ultrasound, a LIVU thermal treatment plan is developed, including the location and spacing of the US emitters and the depth of the tissue treated. A template is used to mark the locations for treatment for both patient and clinician agreement and comfort level.

Ultrasound LIVU energy may be able to have the capacity to change ultrasonic wave emitter pattern. Just like how there are different lobe heads for different purposes, the device may have a universal probe head with the capacity to emit different pulsatile waves: capacity to automatically calculate different tissue density of the targeted tissue and apply the La place Fourier wave equation to calculate the frequency and time requires to penetrate tissue and apply the heat equation to calculate heat influx and distribution patterns.

Sound wave energy can be converted to heat energy and can be used in tissue therapeutically. The La place transform heat equation can be built into the ultrasound system which is used to calculate the heat energy transferred to the tissue with respect to time. The probe may have at least 2 ultrasound emitters that can be moved while applying therapy and are capable of delivering energy into the target tissue.

Bio Heat transfer equation:

$$\frac{\partial T}{\partial t} = K * {V}^{2} * T - b * T + Q ( t ) * I ( r , z )$$

Here, t is the function of temperature change in degree Celsius in all dimensions.
K is the thermal diffusivity (m2/s).
Q (t) is the function of heating rate in respect to the time due to ultrasound absorption.
I (r,z) is the normalized spatial acoustic intensity distribution profile.
r is the axis perpendicular to beam propagation (transverse)
z is the beam propagation axis (longitudinal)

$$b = \frac{{ω}_{b} * {ρ}_{b} * {C}_{b}}{ρ} * C$$

where, ω_{b} is the blood perfusion rate (mL/sec), ρ_{b} and ρ are the density of blood and tissue and C_{b} and C are the heat capacity of blood and tissue.

Ultrasound properties:

| Medium | Density | U/S speed (m/s) | Acoustic impedance |
|---|---|---|---|
| Air | 1.3 | 330 | 429 |
| Water | 1000 | 1500 | 1.5 log6 |
| Blood | 1060 | 1570 | 1.66 log6 |
| Fat | 925 | 1450 | 1.34 log6 |
| Muscle | 1075 | 1590 | 1.7 log6 |
| Bone(+/−) | 1400-1900 | 4080 | 5.7-7.8 log6 |
| Transducer | 5600 | 5500 | 30.8 log6 |

Calculation on how heat will be diffused to a surface, simplified:

$$\begin{matrix}q = - K * ∇ * T \\ = - K ( i \frac{\partial T}{\partial x} + i \frac{\partial T}{\partial y} + i \frac{\partial T}{\partial z} ) \\ = {q}_{x} i + {q}_{y} j + q - z\end{matrix} k$$

Thus, in some embodiments of LIVU devices, which will operate at lower energy delivered to a larger volume than traditional HIFU to avoid thermal injury and charring, the LIVU devices will have live ultrasound image monitoring with an acoustic impedance alarm that is set to a level at which the subcutaneous tissue is to be only denatured. Therefore, liquification and/or boiling of the target tissue is precluded. The specific target tissue may have an optimized algorithm for thermal treatment with planned “on time”, “off time”, and amplitude (power level) of the LIVU pulse train. Prior to treatment, ultrasound imaging is used to ensure there is an adequate depth of the subcutaneous tissue and to evaluate the tissue being treated. With the pre-treatment ultrasound MHz measurement scale of the target tissue, in some embodiments of the LIVU system, the clinician is able to direct the energy given for that targeted tissue; for example, only treat 5 to 12 MHz for adipose tissue and only concentrate energy from 1 cm below the skin to 4 cm max depth. In some embodiments, the LIVU generator monitors live ultrasound acoustic impedance of the tissue being treated and alerts the clinician when clinically significant acoustic changes have been detected and has a detection algorithm that shuts off energy when a predetermined value of tissue acoustic impedance is met. Devices, systems and methods of the various embodiments are designed and configured to apply ultrasound energy to subcutis tissue to achieve a treatment endpoint such as clinical denaturation, and with the ability to monitor treatment progress and to terminate energy delivery as required to prevent ablation/thermal injury to the patient.

Thermally treating tissue requires that the temperature of the target tissue be raised to a level at which the desired modification occurs. Supplying a unit of energy (Joule) to a volume of tissue will raise the temperature of that tissue as the energy is converted to heat. This occurs whether the energy supplied is RF electricity or high frequency ultrasound. Each unit of energy applied to the tissue raises the tissue temperature commensurately.

The rate at which the temperature of the tissue rises is proportional to the rate at which the energy is input to the tissue. Power (measured in Watts) is the amount of energy transferred or converted per unit time. 1 Watt=1 Joule per second. Increasing the power level of a device (more Watts) decreases the time required to thermally achieve a clinical effect.

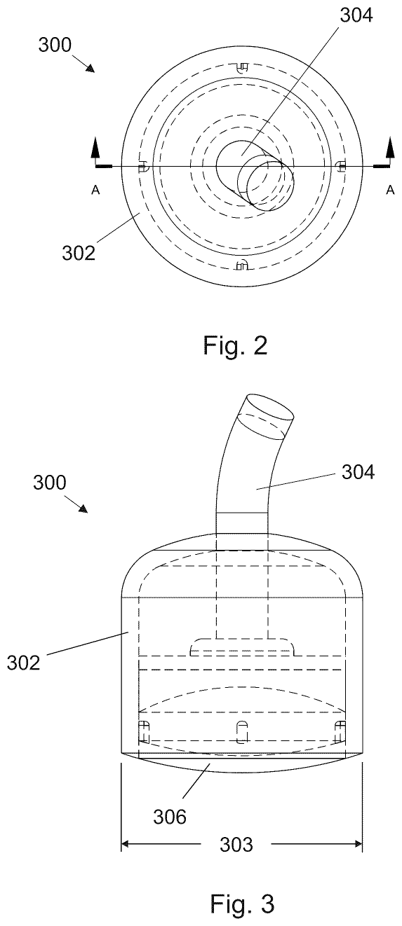

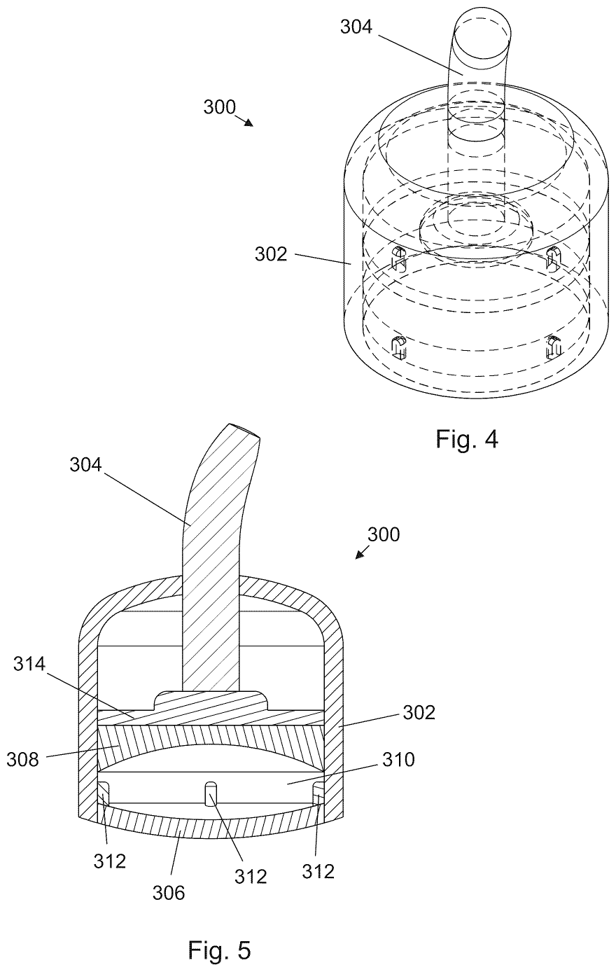

Referring now to FIGS. 2 through 5 depicting elements of a device/system 300 of an illustrative embodiment for treating adipose tissue, device 300 has a housing 302 from which passes cable 304. Cable 304 is connected to and in communication with a source for ultrasonic energy, and with circuitry that controls the characteristics of the supplied energy, and its dispersal as will be subsequently described. As best seen in FIG. 5, transducer 308 is connected by circuitry in coupling 314 to cable 304. Region 310 bounded by the distal face of transducer 308 the proximal surface of lens 306 and circumferentially by housing 302 is filled with a coupling liquid that conducts ultrasonic energy from transducer 308 to lens 306 and therethrough to the treatment site. The coupling liquid also cools transducer 308. In some embodiments the coupling liquid is circulated externally through a cooling system that maintains the liquid at a predetermined temperature to ensure that lens 306 does heat to temperatures that may cause thermal injury. Optical thermal sensors 312 monitor the skin temperature as the device is moved across the skin of a patient. As Optical thermal sensors 312 monitor the skin temperature and each may have an independent energy shut off alarm or other remedial action if the skin temperature raises or lowers significantly. Raising temperatures will shut off to prevent thermal damage and lowering will shut off as it could indicate the probe head not in contact with the skin.

The rate at which tissue temperature increases is strongly affected by the size of the area over which energy is applied to the tissue. This is referred to as “power density” and has the units “Watts/cm²”. Applying 100 Watts to a 2 cm² area of tissue will result in twice the rate of tissue temperature increase compared to applying that 100 Watts to a 4 cm² area of tissue.

By focusing the energy applied to tissue (increasing the power density), high rates of tissue heating can be achieved for small tissue masses. Indeed, virtually instantaneous tissue vaporization can be achieved when power is concentrated or focused on an extremely small area. Conversely, by controlling the power density in a region for thermal treatment, the rate of temperature increase can be tailored to meet specific requirements. This region can extend proximally and distally from the focal plane, the power density at the focal plane being at the higher end of the treatment range, and at the lower end of the treatment range a predetermined distance away. Using LIVU for the thermal treatment, where the power density maximum being applied across the treatment region is maintained at or below 50 W/cm² avoids the risk of ablating the tissue being treated.

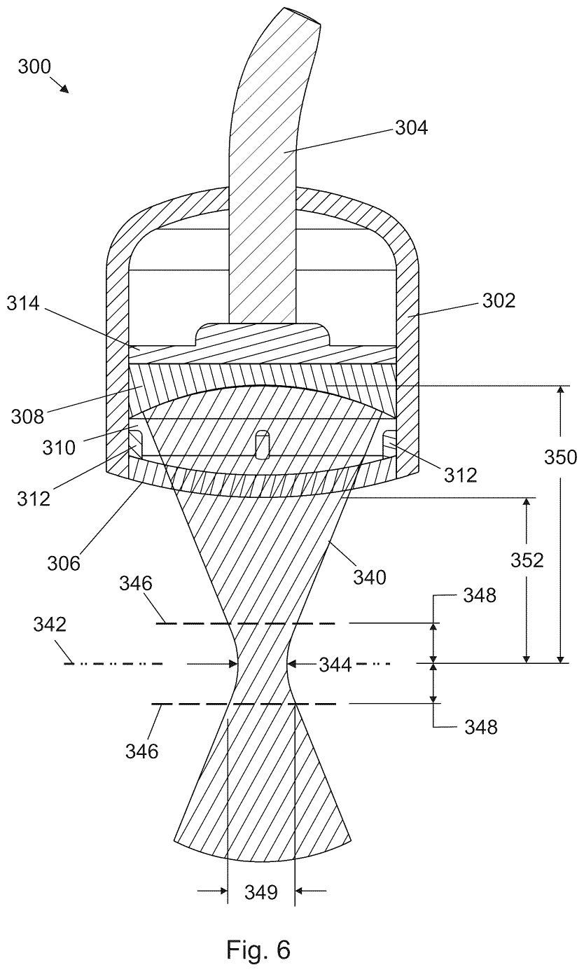

FIG. 6 depicts the distribution of focused ultrasound energy 340 emitted by transducer 308. Transducer 308 has a focal length 350 at which maximum concentration of energy occurs. As seen in FIG. 6, energy 340 from transducer 308 is not highly focused, but rather is defocused to have a minimum diameter 344 at focal plane 342. The power density, and the resulting rate of heating of tissue at the focal plane, is determined by dividing the power output of transducer 308 by the cross-sectional area at focal plane 342. Planes 346 are displaced distance 348 from focal plane 342. The power density at planes 346 and the associated rate of tissue heating is determined by dividing the power output of transducer 308 by the cross-sectional area at planes 346. As depicted in FIG. 6, diameter 349 at planes 346 is approximately 30% greater than diameter 344 at focal plane 342. The increased cross-sectional area at planes 346 decreases the power density by approximately 40% since it is an area calculation determined by the diameter difference squared. In devices of the various embodiments, through selection of the transducer diameter, focal length and degree of de-focusing, a “treatment zone” is created that extends symmetrically focal plane 342 predetermined distance 348. Within this zone, the power density is sufficient to achieve a desired clinical effect (denaturing of adipose tissue) in a predetermined treatment time, without unwanted tissue effects caused by overheating of tissue at focal plane 342 where maximum power density occurs.

Transducer 308 may be formed of a single element wherein the focal length and degree of focusing are fixed. In other embodiments, transducer 308 is formed of multiple elements connected to a controller. These elements may form a “phased array” transducer in which the focal length and focusing may be modified by delaying the energy pulse from individual elements so that they all arrive at the focal point at the same time. In some embodiments incorporating multi-elements transducers, the focal length and focus characteristics may be varied in a pre-determined, programmed manner during use to increase the size of the treatment zone.

In some embodiments transducer 308 may function as an ultrasonic imaging transducer as well as a generator of ultrasonic treatment energy so as to provide real-time imaging of tissue undergoing treatment thereby allowing a clinician to monitor the process. Alternatively, other imaging may be used. Among these are fluoroscopy, CT scanning, or Magnetic Resonance Imaging (MRI). In some embodiments images are displayed on a screen integral with or mounted to the device handpiece. In others the image may be displayed remotely but within the field of vision of the clinician so as to allow direct monitoring of the thermal treatment process.

As previously described, the time required for tissue within this treatment zone to reach the treatment temperature is determined by the design of the system, specifically the power output and the focusing characteristics of the transducer. In devices and systems of the various embodiments configured for transdermal treatment of adipose tissue, the device is not stationary, but rather is moved over the tissue in a predetermined path so as to thermally treat a region of target tissue. The temperature achieved by tissue within this region is determined by factors previously herein described, and by the speed at which the treatment device is traversed over the tissue. In some embodiments, real time imaging of the treatment site allows the clinician to monitor progress of the treatment on tissue therein so that the clinician can adjust the speed of travel to achieve the desired results.

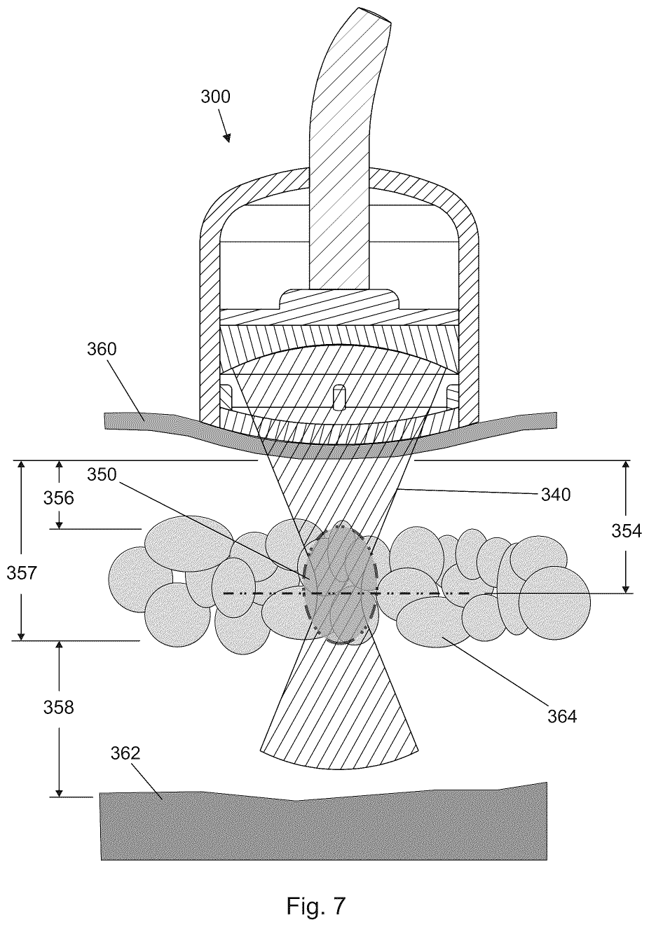

FIG. 7 depicts a mass of fat lobules (adipose tissue) 364 undergoing thermal treatment in accordance with principles disclosed herein. Tissue 364 is centered distance 354 below skin 360 placing it at the focal plane of focused energy region 340. Its proximal margin is distance 356 below skin 360, and its distal region is displaced from fascia/muscle 362 distance 358. Treatment zone 350 is centered axially within mass of adipose tissue 364. Prior to treatment, the region to undergo treatment is evaluated using ultrasound or another imaging method to ensure that distances 356 and 358 fall within predetermined bounds that ensure safe treatment of lobules 364 without thermal damage to muscle 362 or skin 360. In some embodiments distance 356 is at least one centimeter and distance 357 is no more than four centimeters, and distance 358 is at least one centimeter. Device 300 is moved across the skin at a rate that energy within treatment zone 350 raises the temperature of adipose tissue 364 to a value at which denaturing of tissue 364 occurs. The ultrasound imaging head 308 will be connected to the generator to monitor ultrasound acoustic impedance of the tissue zone 350 being treated and have a mechanism of providing a remedial action such as an alarm that shuts off energy once tissue acoustic impedance is met such as by changes to tissue density or elasticity; therefore, energy would be run though the subcutis tissue and automatically shuts off energy when tissue is treated to be denatured only.

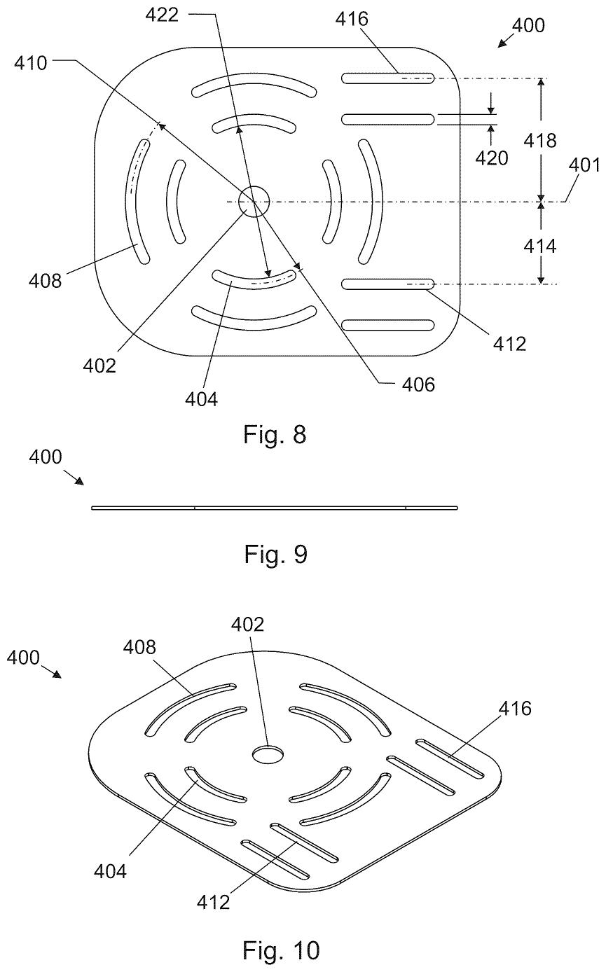

FIGS. 8 through 10 depict a template 400 for use with devices and systems of the various embodiments. Template 400 aids in positioning of handpiece 300 prior to, and during treatment of a patient. Template 400 has a circular central opening 410, first radial slots 404 of radius 406, second radial slots 408 of radius 410, first linear slots 412 positioned distance 414 from centerline 401, and second linear slots 416 positioned distance 418 from centerline 401. Distance 414 is equal to radius 404 of first radial slots 404 and distance 418 is equal to radius 410 of second radial slots. Slots 404, 408, 412 and 416 have a common width 420. Radius 406 and width 420 are chosen such that the circular region bounded by first radial slots 404 has a diameter 422 that is equal to diameter 303 of housing 302 of device 300.

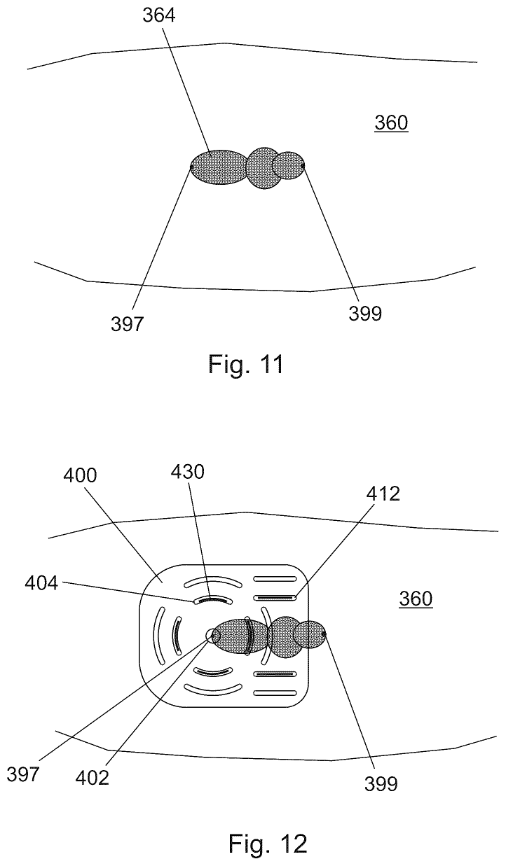

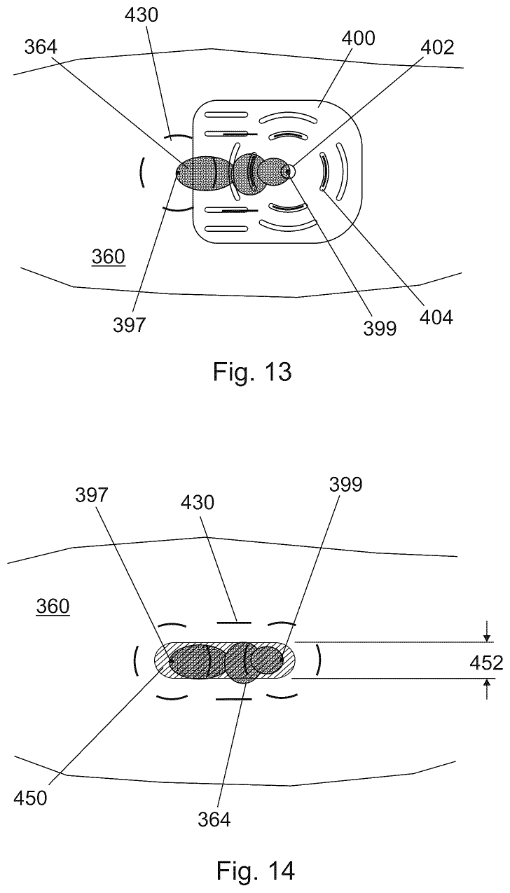

Template 400 may be used as depicted in FIGS. 11 through 16. FIG. 11 depicts subcutaneous fat lobules 364 beneath dermis 360. In preparation for treatment, the clinician places first mark 397 at a first end of the collection of fat lobules 364 and a second mark 399 at a second end. Template 400 is positioned as shown in FIG. 12 with first mark 397 centered in opening 402 of template 400 and the portion of template with linear slots 412 pointed toward second mark 399. Using a suitable marker, the clinician makes locating lines 430 using radial slots 404 and linear slots 412. Thereafter template 400 is repositioned as depicted in FIG. 13 with second mark 399 centered in opening 402. The clinician makes additional locating lines 430 using radial slots 404 so as to define a region bounded by locating lines 430 within device 300 will move linearly from an initial location centered on first mark 397 to a terminal position centered on second mark 399. Treatment begins with positioning device 300 at the first end of the bounded region within radial locating lines 430. Device 300 is energized and moved at a controlled rate to the second end of the region. Doing so creates a region of treatment 450 of width 452 within which the energy density is sufficient to achieve a desired clinical effect, in this example being denaturing of fat lobules 364. Width 452 is not constant but rather is determined by focusing characteristics of device 300, the distance from the focal plane, and the speed at which device 300 is moved across dermis 360, in the manner previously described with reference to FIGS. 6 and 7.

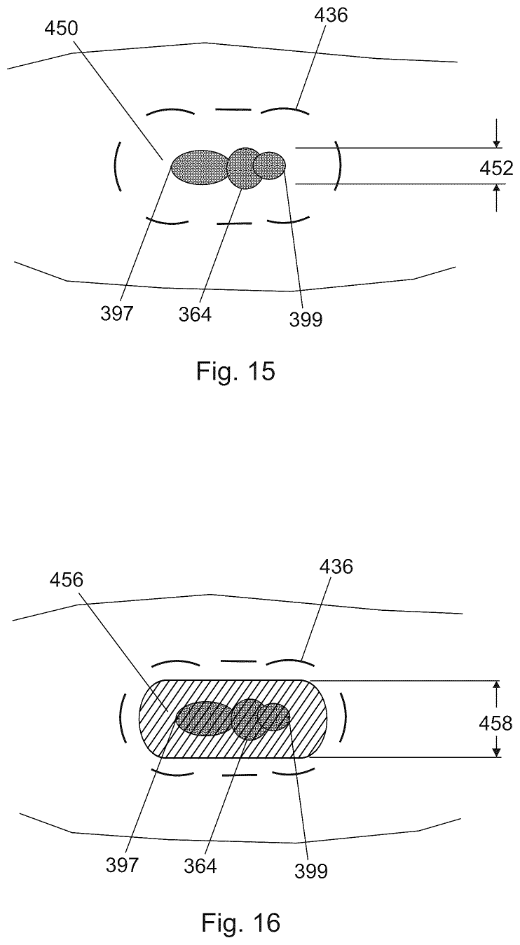

Width 452 of treatment region 450 produced by a single pass may be insufficient to treat larger regions of tissue. FIGS. 15 and 16 depict a method by which template 400 may be used to define a larger treatment region. Locating lines 436 have been formed using radial slots 408 and linear slots 416 of template 400. During treatment, device 300 is moved along a path in which the circumferential surface of device 300 is in close proximity to locating lines 430 so as to create treatment region 456 of width 458.

Transdermal treatment systems of the various embodiments achieve clinical effect by subjecting tissue to focused ultrasonic energy created by one or more transducers, the transducers converting pulses of high frequency electrical energy to high frequency mechanical energy that is focused on the treatment region. In some embodiments treatment is monitored visually using ultrasonic imaging. In ultrasonic imaging transducers convert pulses of high frequency electrical energy to high frequency mechanical energy that is focused in the treatment region. Imaging transducers have the ability to convert ultrasonic energy reflected by tissue and structures in the treatment region into electrical energy. This reflected energy is processed into images that are displayed on a screen. Systems of the of the various embodiments may include transducers for both tissue treatment and for imaging thereby allowing the clinician to monitor progress of the treatment and terminate the process when treatment is complete.

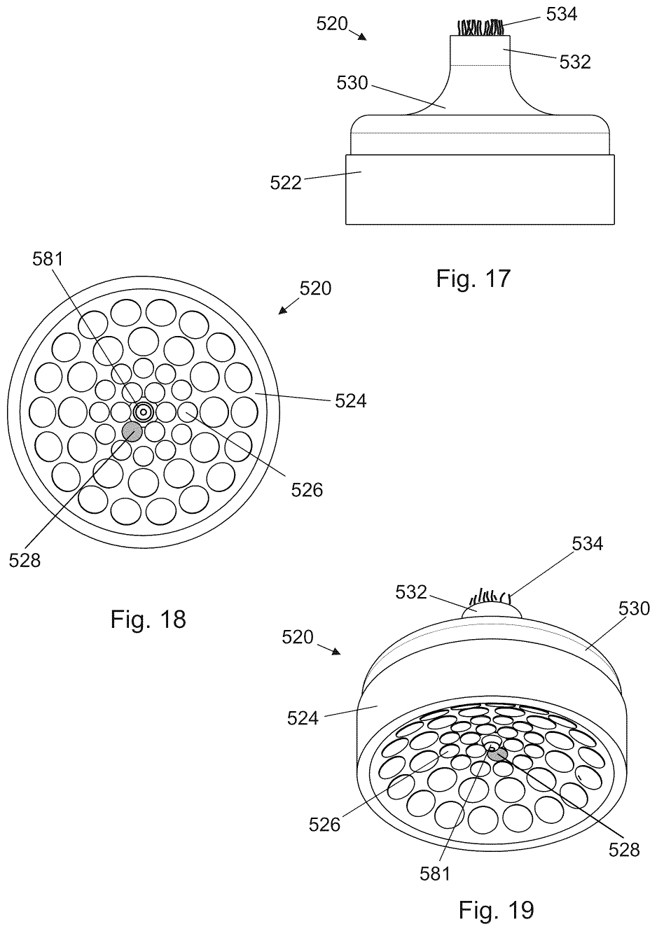

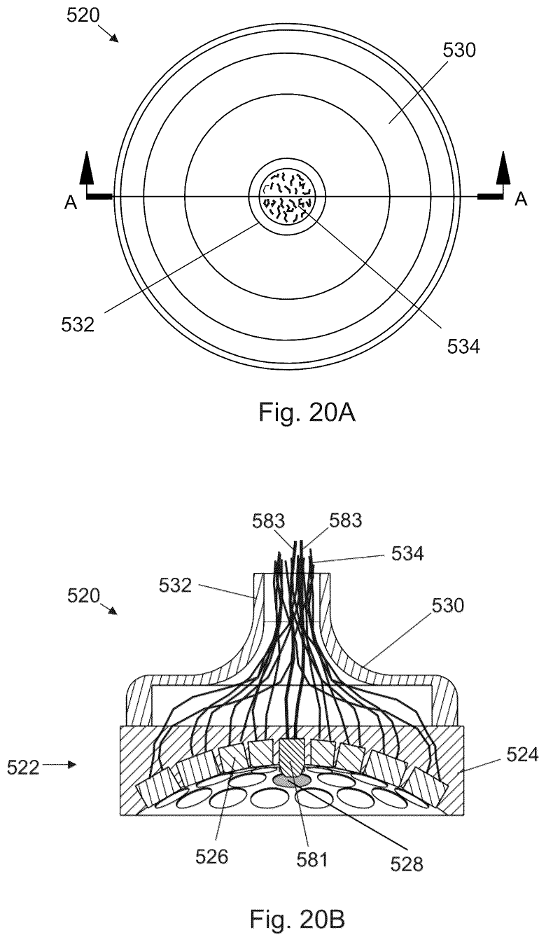

Transducers used in devices and methods of the various embodiments may be formed of a single element with focusing characteristics determined solely by its elliptical transmitting surface, or by an array of multiple transducing elements wherein delays are applied to pulses of energy supplied to the individual transducing elements so that the pulses from the multiple elements arrive at a focal point simultaneously. These phased array transducers may be flat, depending solely on timing of the pulses to achieve focused energy, or may have the elements arranged in an elliptical shape such that focusing is achieved by the shape combined and delays applied to the energy pulses. In some embodiments a transducer assembly with multiple emitter head elements arranged in a parabolic or other shape is used. An exemplary transducer assembly of this type is depicted in FIGS. 17 through 20B. Transducer assembly 520 has a distal portion 522 comprising element 524 in which are mounted emitter head elements 526 and temperature sensor 581. 524 is the housing holding the mounted emitter head elements 526, central imaging transducer 528 and temperature sensor 581. Transducer assembly 520 has a proximal portion formed of housing 530 with tubular proximal portion 532 from which pass wires 534 from emitter heads 526, and wires from temperature sensor 581. Transducer assembly 520 has multiple emitter head elements mounted in a concave elliptical configuration. Transducer assembly 520 has a first centrally located transducing imaging element 528 and a second array of emitting treatment elements 526. Element 528 provides ultrasound visualization during treatment. Elements 526 provide therapeutic ultrasound energy.

Transducer 520 (FIGS. 17 through 21) is a phased array transducer allowing the focusing characteristics of the transducer to be modified as required for optimal treatment of a subcutaneous tissue. These characteristics may include the focal distance of transducer 520 and the diameter of the energy beam at the focal plane as described in FIG. 6 and descriptions associated therewith. In some embodiments, transducer 520 has a mean focal distance imparted by the parabolic positioning of emitter head elements 526, the focal distance falling on the mean between the minimum and maximum treatment distances below the dermis. By adjustment of the delays of pulses to emitter head elements 526, the focal length can be increased or decreased such that the focal plane is centered within the tissue to be treated.

The location of imaging transducer element 528, and the number of treatment transducer elements 526 may vary from one embodiment to the next. This shape, the number and the positioning of elements 526 and 528 may be optimized to achieve specific focusing characteristics.

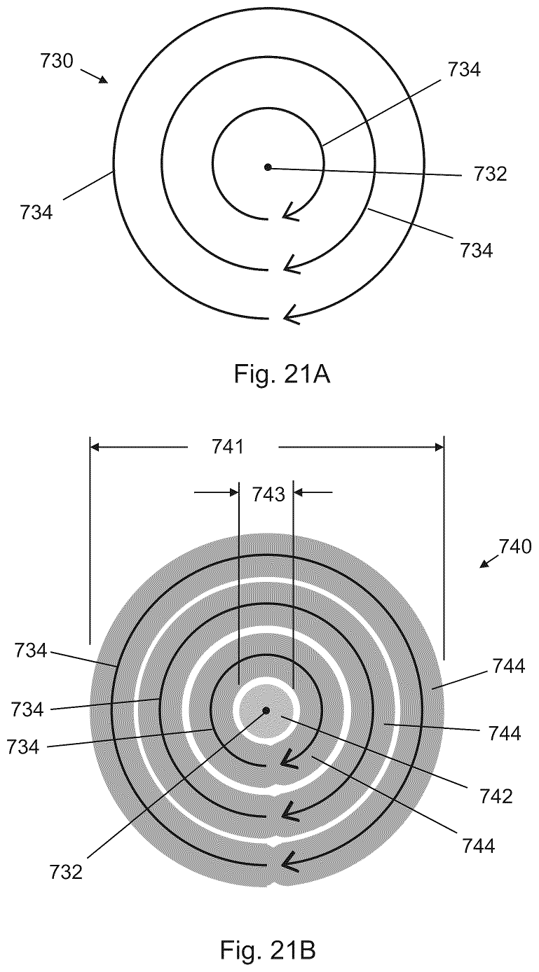

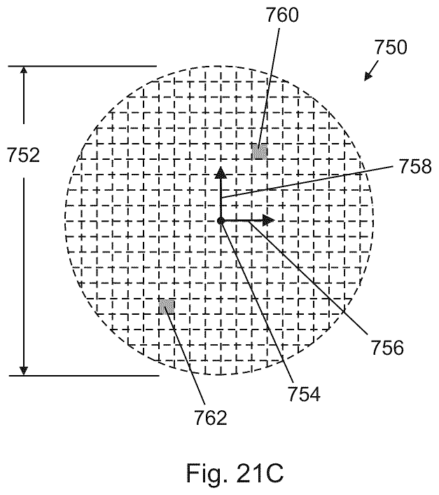

The use of phased array transducers allows modification of the location of the focal point (region of highest energy density) within a focal plane. The focal point may be moved from a first location to a second location by suitably modifying the time delays to individual transducer elements making up the array. This allows the focal point to be directed along a predetermined path so as to treat a region larger than would be possible when the focal point is maintained at a fixed location within the focal plane. For instance, FIG. 21A depicts a path 730 that the focal point of a phased array transducer assembly may follow in creating a treatment region 740 (see FIG. 21B). Path 730 has an initial central point 732 that may be treated, and concentric paths 734. The focal area 742 of the transducer assembly has diameter 743. When moved along path 730, treatment region 740 of diameter 741 is created, regions 744 of treated tissue surrounding concentric paths 734.

In contrast, when using a single element transducer as previously described and shown in FIGS. 2 through 5, the diameter of the treatment region (region of high energy density at the focal plane) is determined solely by the fixed focusing characteristics of the transducer. Referring to FIG. 6, diameter 344 at focal plane 342 is determined by the characteristics of transducer 308.

As previously stated, the use of phased array transducers allows modification of the location of the focal point (region of highest energy density) within a focal plane. The focal distance of the transducer assembly can also be controlled by adjusting the delay applied by each transducer element. By varying the focal length of a transducer assembly and traveling the focal point along a predetermined path (730 in FIG. 21A and 740 in 21B) a treatment volume can be created. In some embodiments, transducer assembly 520 is connected to a control system that allows the focal point to travel along a predetermined path as depicted in FIGS. 21A and 21B, and to control and vary the focal distance as previously described. This allows the creation of treatment volumes (or regions) that may be optimized for specific conditions and tissue types.

The distance that a focal point in the focal plane may be displaced from the center of the plane is limited, the portion of the plane within this radius is the usable portion. In some embodiments a coordinate system is established within this working area for use when directing the travel of the focal point within this working area. FIG. 21C depicts a working zone 750 of diameter 752 wherein is formed a grid with an X-Y coordinate system about center point 754. Locations within the grid are specified by coordinates with reference to X axis 756 and Y axis 758. For instance location 760 has coordinates (3,4) and location 762 has coordinates (−4,−6). A database image can be created in which is recorded the unique combination of the ultrasound signal characteristics to the imaging transducer element 528 required to bring the focal point for treatment to each location. Using suitable control software, a sequence of X-Y coordinates to which the focal point will move can be sent to transducer assembly 520 using the corresponding unique combination of signals to imaging transducer element 528 recorded in the database. The precision of the location placement is determined by the resolution of the grid 750 of the coordinate system of FIG. 21C. Phased array transducer assembly 520 also has the ability to increase or decrease the focal distance of transducer assembly 520. Accordingly, a sequence of beam positions may be executed on a first focal plane at a first focal distance, and then repeated on a second focal plane at a second focal distance so as to create a portion of a treatment volume. Alternatively, each focal point location in a sequence may be specified using an X-Y-Z coordinate system in which the Z dimension is the focal length.

The shape of a desired treatment zone may be created as a solid model using a Computer Aided Design (CAD) system. A file describing the model geometry is output to a post processor program which creates focal planes and paths on those planes that will result in treatment of tissue within that volume. These planes and paths are created using geometric information from the CAD file. Additionally, values for diameter 742 of the energy field at the focal plane (minimum diameter 344 at focal plane 342 in FIG. 6) and treatment limits of phased array transducer assembly 520. The output of the post processor is then converted to a sequence of focal point positions as previously described using the previously created database.

The configuration of a treatment zone is determined by the geometry of a solid model created in the CAD system allowing a high degree of flexibility and optimization for specific conditions. In other embodiments, the geometry of a structure to be treated may be determined through imaging and a solid model created to approximate this geometry.

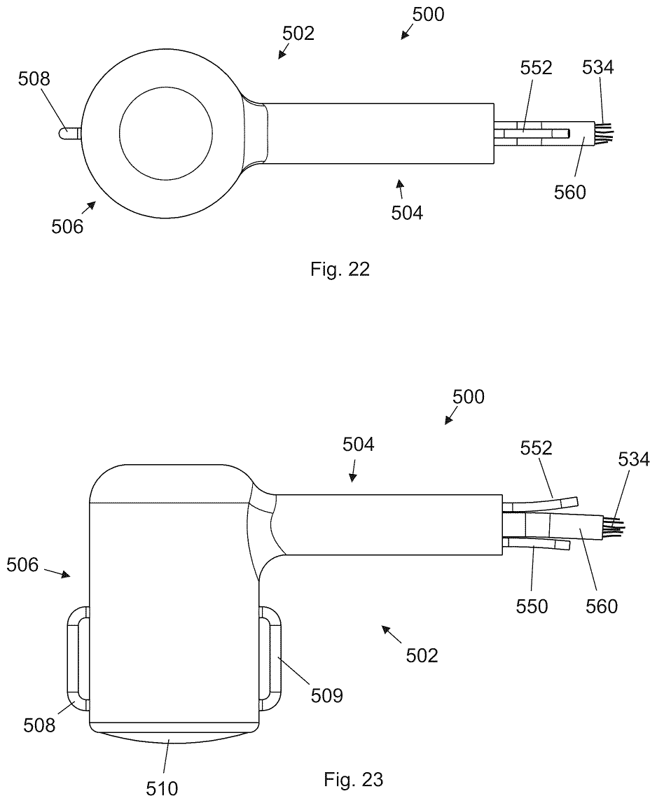

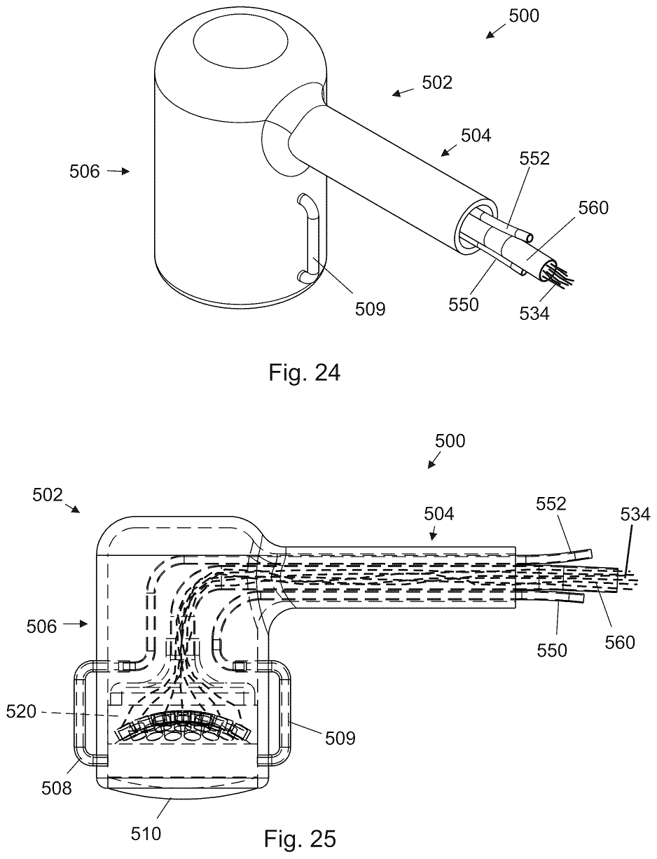

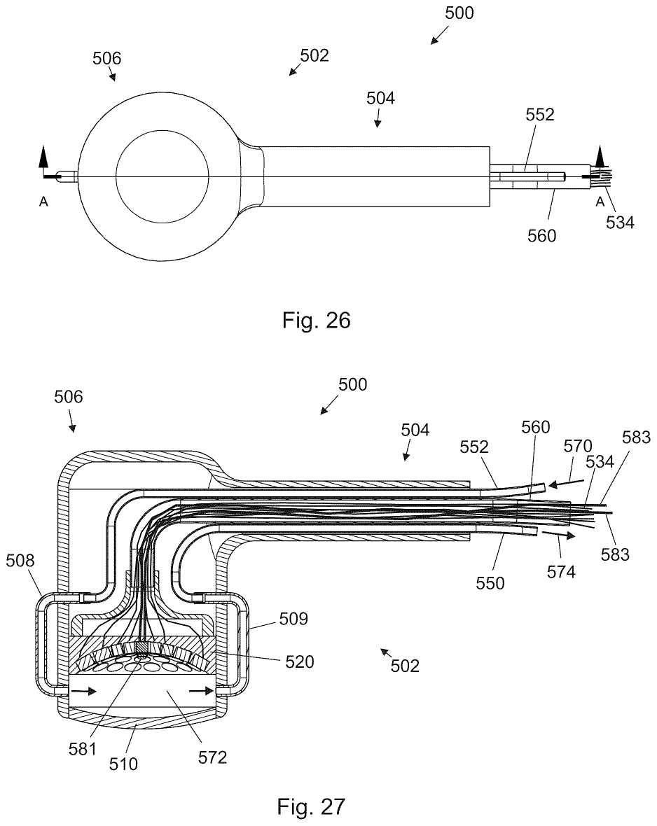

FIGS. 22 through 27 depict the handpiece 500 of a LIVU treatment system constructed in accordance with the principles of the disclosure. Handpiece 500 has a housing 502 with a distal portion 504 containing transducer 520 and connective elements 534 connected thereto, and a proximal handle portion 504, from which exit via its proximal connective elements 534 and 583 contained within flexible tubular member 560, and tubular elements 550 and 552. Region 572 is filled with a coupling fluid. Temperature sensor 581 is used to monitor the temperature of the coupling liquid and indirectly window 510 in contact therewith to ensure that the temperature of the coupling fluid and window 510 do not reach temperatures that could thermal injury to dermal tissue in contact with window 510. Tubular elements 508 and 509 are affixed respectively to distal and proximal portions of distal portion 506 of housing 502. As best seen in FIG. 27, inflow tubular element 552 together with distal tubular element 508 provide an inflow 570 path for coupling fluid to region 572 bounded by distal portion 506 of housing 500, transducer 520, and window 510. Proximal tubular element 509 together with outflow tubular element 550 provide a return 574 for coupling fluid from region 572. During use, coupling fluid is circulated between region 572 and a remotely located cooling means wherein the temperature of the fluid is also monitored and cooled if the temperature is above a predetermined value.

In other embodiments, coupling fluid is not circulated externally as in handpiece 500. In these embodiments the rate of heating of the coupling fluid by transducer 520 is insufficient to raise the fluid temperature above that which may cause discomfort or thermal injury to a patient's skin against which window 510 is pressed during treatment. The temperature of the coupling fluid is monitored using temperature sensor 581 and if the temperature exceeds a predetermined value an alarm message is displayed for the clinician and treatment is interrupted until the fluid temperature decreases below the set value. The rate of heating is determined by the power required to achieve the desired clinical effect. Factors affecting the power requirements are previously herein described with reference to FIG. 6 and associated text.

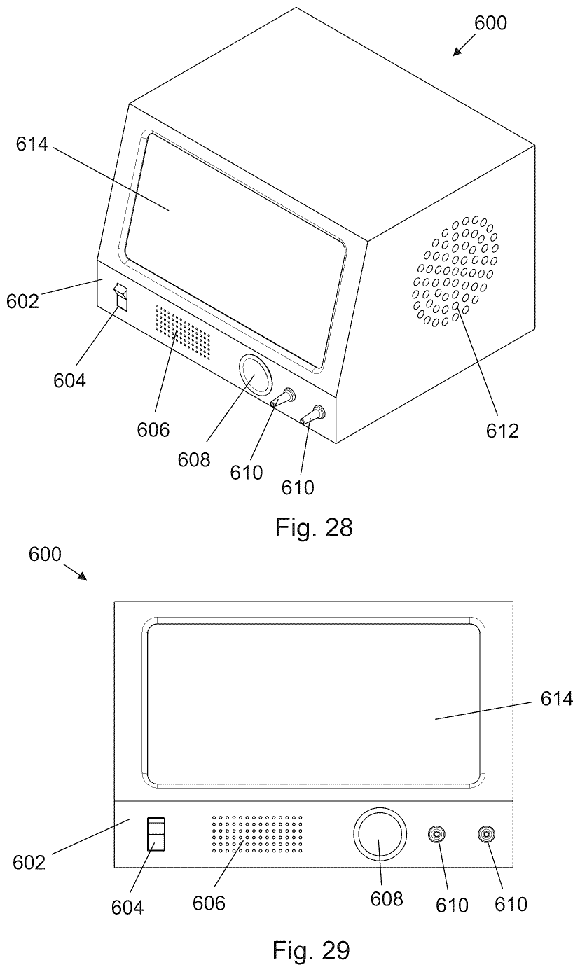

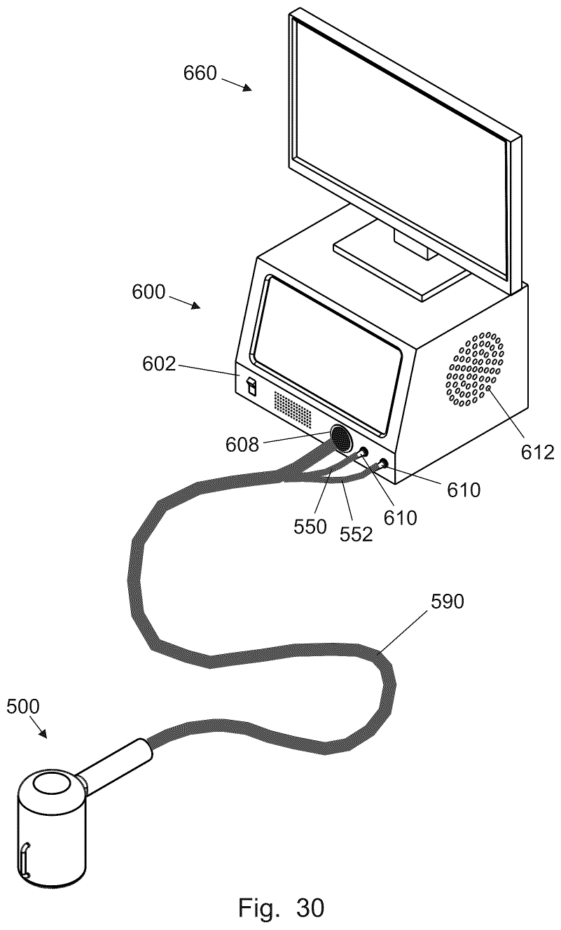

Control console 600 of the illustrative embodiment, depicted in FIGS. 28 through 30, has a front panel 602 wherein is located power switch 604, perforations 606, receptacle 608 for connection to handpiece 500 by cable assembly 590, and tubing connectors 610. Console 600 has perforations 612 formed in a first wall its enclosure. On the opposing, second wall an opening (not shown) with a suitable filter provides an inflow path for air driven by a fan inside the enclosure adjacent to perforations 612, perforations 612 providing an outflow path. Airflow through console 600 removes waste heat generated by the power supply contained within console 600. In some embodiments in which coupling fluid is circulated to handpiece 500, a heat exchanger is positioned in the flow path, with inflow and outflow via tubular elements 550 and 552 connected to tubing connectors 610. In some embodiments the cooling flow is controlled by a thermostat in the fluid flow path. Perforations 606 provide a path for audio signals from inside console 600. These may include a tone that sounds during activation, and tones that sound to alert the operator to an error condition, prompting the operator to check display 614 for specific related messages. In some embodiments display 614 is a touch screen that may be used for operator input and for display of process information. Monitor 660 displays all treatment and safety features for the operator including imaging of the treatment site acquired by the ultrasound imaging transducer 528 and temperature readings of the optical sensors 312 of the probe head assembly 520.

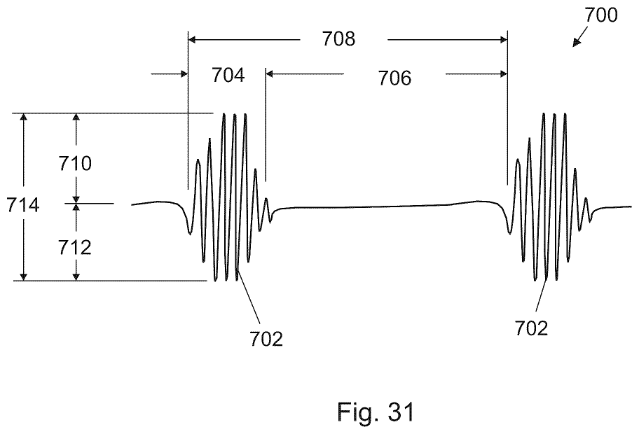

FIG. 31 depicts a pulsed output waveform 700 of ultrasonic energy. Waveform 700 has pulses 702 with an on-time 704 separated by off-time 706 and a repetition period 708. Energy in pulses 702 have a frequency suited to coupling with tissue of the type to be treated. Pulses 702 have a peak-to-peak amplitude 714 formed of positive amplitude 710 and negative amplitude 712. Each pulse 702 contains a predetermined amount of energy which will commensurately increase the temperature of a unit of tissue with which it interacts. The rate at which tissue can be heated, then, is determined by the amount of energy in each pulse (determined by pulse amplitude 714 and on-time) 704, and the rate at which pulses are supplied (determined by off-time). The rate at which energy is supplied is referred to as power.

FIG. 31 demonstrates an “on time”, “off time”, and amplitude (power level) of an ultrasound pulse train with the y-axis would be the voltage and the x-axis would be the time in microseconds. The generator or operating clinician can change the energy in voltage and the wavelength time. An algorithm can be used to treat subcutaneous tissue built into the generator. The pulse train goes through the skin into the subcutaneous tissue where it then meets a treatment point located between 10 to 40 mm depth and above the fascia. The clinician can alter the angle of the transducer depending upon the patient's treatment plan.

When treating tissue with a pulsed (interrupted) waveform, energy flows into the tissue during the on-time and flows from the heated tissue into surrounding tissue during the off time. To achieve tissue temperatures required for a desired clinical effect, the rate of energy input to the tissue must be greater than the rate of heat loss to the surrounding tissue. The volume of tissue into which the power is input also determines the rate of tissue heating, and the power required to achieve temperatures required for a desired clinical effect. As previously described with regard to FIGS. 6 and 7, the volume of tissue to be treated in methods of the various embodiments is determined by focusing characteristics of the transducer. FIG. 7 exhibits ultrasonic wave emitter patterns to focus on a designated treatment focal point or region inside the body. Transducers can utilize wide angles of ultrasonic pulses to produce a focused amount of energy within the body. The clinician/generator can use alternate patterns or shapes to optimize patient care. The probe head may have multiple ultrasonic emitter heads (transducer element) focused on the same target point as demonstrated in FIGS. 18 and 19. The number of emitter head elements is may vary from one embodiment to the next. The emitter heads will be able to only focus LIVU from a minimum to a maximum, such as from 1 to 4 cm of depth 354.

System 600 allows the operator to visually monitor treatment and adjust aspects of the ultrasound energy output. For instance, in the absence of, or slow rate of clinical effect, the energy level may be increased by decreasing the off-time, increasing the on-time, or increasing the amplitude of the energy supplied. Conversely, a high rate of heating of target tissue may create the possibility of overtreatment, the target tissue being subjected to unintendedly high temperatures causing undesired tissue effects. The rate of energy input to the treatment site may be decreased by adjustment of on-time, off-time and energy amplitude. Systems of the various embodiments provide the clinician with means and methods for optimizing the treatment parameters.

Embodiments are anticipated in which the optimization previously described, wherein optimizing of focusing and energy characteristics are automatically performed based on aspects of the imaging of the treatment site according to programmed methodology.

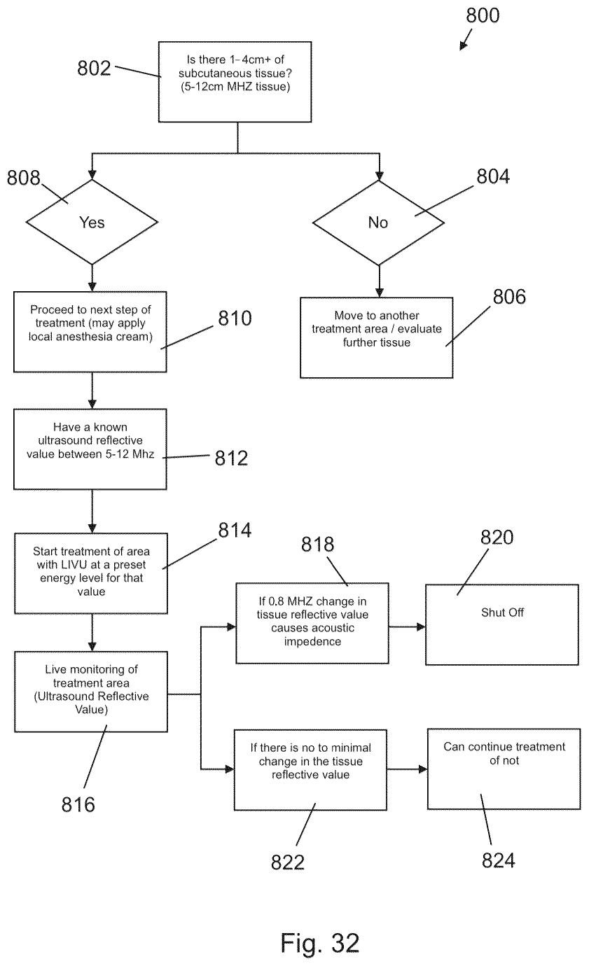

FIG. 32 depicts the planning and treatment algorithm 800 for LIVU in the denaturing of subcutaneous tissue. The preoperative evaluation of the subcutaneous tissue by ultrasound 802 from the minimum to the maximum, such as 1-4+ cm, below the skin will be done. If the ultrasound reflective value of the tissue at the minimum to maximum, e.g., 1-4+ cm, is not in the 804 in the 5-12 MHz range the treatment area will be 806 changed or more in-depth evaluation of the tissue is done. If the preoperative ultrasound evaluation of the tissue at the minimum to maximum, e.g., 1-4+ cm, of depth is in the 808 5-12 MHz range, then can proceed to 810 the next step in the treatment and local anesthesia can be applied.

With having a known reflective value of the tissue 812 between 5-12 MHz, the treatment with LIVU 814 for denaturing the subcutaneous tissue can be started at a preset energy delivery for that know value. Live ultrasound imaging of the LIVU treatment area 816 is done monitoring any changes in the reflective value. If during ultrasound monitoring the live LIVU tissue treatment the reflective value changes 818 by 0.8 MHz or more, an acoustic impedance alarm 820 will automatically shut off the energy delivery and/or take other remedial action. If during ultrasound monitoring the live LIVU tissue treatment the reflective value changes 822 are less than 0.8 MHz, then the treatment 824 can be continued per the clinician and the acoustic impedance alarm is not activated.

### Elements of the Exemplary Embodiments

In the context of the various embodiments disclosed herein, the following definitions apply:

The words “a”, “an”, and “the” as used herein mean “at least one” unless otherwise specifically indicated.

Devices and methods of the of the various embodiments thermally treat tissue using ultrasonic energy supplied by a transducer that converts electrical signals to ultrasonic mechanical oscillations thereby producing ultrasound energy. This ultrasonic energy is focused by the transducer. Herein the transducer supplying therapeutic ultrasound energy may be referred to variously as a “transducer assembly, transmitter, or emitter”.

Transducers may be formed of a single element wherein the focal length and degree of focusing are fixed. In other embodiments, transducers are formed of multiple transducer elements connected to a controller. These elements may form a “phased array” transducer in which the focal length and focusing may be modified by delaying the energy pulse from individual elements so that they all arrive at the focal point at the same time. In some embodiments incorporating multi-element transducers, the focal length and focus characteristics may be varied in a pre-determined, programmed manner during use to increase the size of the treatment zone. In some embodiments the transducer may function as an ultrasonic imaging transducer as well as a generator of ultrasonic treatment energy so as to provide real-time imaging of tissue undergoing treatment thereby allowing a clinician to monitor the process. Elements forming a phased array transducer may be interchangeably referred to as “emitters”, “emitting elements” or “transducer elements”.

The term “proximal” refers to that end or portion which is situated closest to the user; in other words, the proximal end of an ultrasound-surgical device of the various embodiments will typically include the handle portion.

The term “distal” refers to that end or portion situated farthest away from the user; in other words, the distal end of an ultrasound-surgical instrument of the various embodiments will typically include the ultrasound emitter/imaging portions.

The disclosure refers to the thermal treatment of tissue. As used herein, the term “tissue” refers to biological tissues, generally defined as a collection of interconnected cells that perform a similar function within an organism. The present disclosure is not limited in terms of the tissue types to be treated but rather has broad application to the thermal treatment of any target tissue with particular applicability to the denaturation or desiccation of subcutaneous tissue.

The term “denature” or “denaturation” as used herein refers to the causation of cell lysis, without breakdown of the bonds between cells, minimal liquefaction and no charring. Cell membranes could be intact but internal components are disrupted. Denatured tissue is absorbed by the body after treatment.

As used herein the term “ablation” refers to non-destructive thermal treatment of tissue using ultrasound energy for the purpose of denaturation or desiccation.

The embodiments have both human medical and veterinary applications. Accordingly, the terms “subject” and “patient” are used interchangeably herein to refer to the person or animal being treated or examined. Exemplary animals include house pets, farm animals, and zoo animals, especially mammals.

Unless otherwise defined, all technical and scientific terms used herein have the same meaning as commonly understood by one of ordinary skill in the art to which this disclosure belongs. In case of conflict, the present specification, including definitions, will control.

As can be appreciated from the description above, embodiments have provided various features that allow for LIVU treatment of subcutaneous tissue. Various risk factors have been addressed by the corresponding various features.

While embodiments have been particularly shown and described, it will be understood by those skilled in the art that various other changes in the form and details may be made therein without departing from the spirit and scope of the treatment.

What is claimed is:

1. A method of treating subcutaneous tissue, comprising:
applying low intensity volumetric ultrasound to the subcutaneous tissue at an area to be treated; and
continuing the application of the low intensity volumetric ultrasound until the subcutaneous tissue at the area to be treated has achieved a treatment endpoint.

2. The method of claim 1, further comprising:
performing an ultrasound analysis at a subcutaneous depth over an area to be treated to develop a treatment plan; and
applying low intensity volumetric ultrasound into the subcutaneous tissue at the area to be treated according to the treatment plan.

3. The method of claim 1, further comprising:
monitoring acoustic impedance to determine when the subcutaneous tissue at the area to be treated has met the treatment endpoint.

4. The method of claim 1, further comprising:
performing real-time imaging to monitor the subcutaneous tissue to be treated to determine when the subcutaneous tissue at the area to be treated has achieved the treatment endpoint.

5. The method of claim 1, further comprising:
monitoring the temperature of skin of a patient over the area containing the subcutaneous tissue to be treated to determine when the subcutaneous tissue at the area to be treated has met the treatment endpoint.

6. The method of claim 1, further comprising:
utilizing a template to mark skin of a patient over the area to be treated; and
applying low intensity volumetric ultrasound into the subcutaneous tissue at the area to be treated based on marks on the skin.

7. The method of claim 1, further comprising:
applying low intensity volumetric ultrasound into the subcutaneous tissue at the area to be treated using phased array ultrasonic transducer with an ability to modify focusing characteristics during use so as to create a predetermined treatment volume.

8. The method of claim 1, further comprising:
adjusting power of a low intensity volumetric ultrasound device to set a depth of treatment to be that of subcutaneous tissue at the area to be treated.

9. The method of claim 1, further comprising:
applying low intensity volumetric ultrasound as pulse trains into subcutaneous tissue at the area to be treated; and
continuing the application of the pulse trains until the subcutaneous tissue at the area to be treated has achieved the treatment endpoint.

10. The method of claim 1, further comprising:
adjusting a wave emitter pattern of a low intensity volumetric ultrasound device to set a depth of treatment to be that of subcutaneous tissue at the area to be treated; and
applying, by the low intensity volumetric ultrasound device with the adjusted wave emitter pattern, low intensity volumetric ultrasound into the subcutaneous tissue at the area to be treated until the subcutaneous tissue at the area to be treated has achieved the treatment endpoint.

11. The method of claim 1, comprising:
applying a local anesthesia to skin of the patient over the area to be treated; and
applying low intensity volumetric ultrasound through the skin where the local anesthesia is applied and into the subcutaneous tissue at the area to be treated until the subcutaneous tissue at the area to be treated has achieved the treatment endpoint.

12. The method of claim 1, comprising:
applying low intensity volumetric ultrasound with a power density level at or below 50 W/cm².

13. The method of claim 1, comprising:
monitoring a physiological parameter while applying the low intensity volumetric ultrasound; and
upon detecting that the physiological parameter meets a threshold, taking a remedial action.

14. The method of claim 13, wherein the remedial action comprises at least one of stopping the applying of the low intensity volumetric ultrasound and generating an alarm.

15. The method of claim 1, wherein the treatment endpoint comprises the subcutaneous tissue being treated becoming denatured.

16. A system that treats subcutaneous tissue, comprising:
an ultrasound device configured to apply low intensity volumetric ultrasound to the subcutaneous tissue at an area to be treated, wherein the ultrasound device continues the application of the low intensity volumetric ultrasound until the subcutaneous tissue at the area to be treated has achieved a treatment endpoint.

17. The system of claim 16, wherein the ultrasound device performs an ultrasound analysis at a subcutaneous depth over the area to be treated to develop a treatment plan and applies low intensity volumetric ultrasound into the subcutaneous tissue at the area to be treated according to the treatment plan.

18. The system of claim 16, wherein the ultrasound device monitors acoustic impedance to determine when the subcutaneous tissue at the area to be treated has met the treatment endpoint.

19. The system of claim 16, wherein the ultrasound device performs real-time imaging to monitor the subcutaneous tissue to be treated to determine when the subcutaneous tissue at the area to be treated has achieved the treatment endpoint.

20. The system of claim 16, wherein the ultrasound device monitors a temperature of skin of a patient over the area containing the subcutaneous tissue to be treated to determine when the subcutaneous tissue at the area to be treated has met the treatment endpoint.

21. The system of claim 16, further comprising a template to mark skin of a patient over the area to be treated such that the ultrasound device applies low intensity volumetric ultrasound into the subcutaneous tissue at the area to be treated based on marks on the skin.

22. The system of claim 16, wherein the ultrasound device applies low intensity volumetric ultrasound into the subcutaneous tissue at the area to be treated using phased array ultrasonic transducer with an ability to modify focusing characteristics during use so as to create a predetermined treatment volume.

23. The system of claim 16, wherein the ultrasound device adjusts power to set a depth of treatment to be that of subcutaneous tissue at the area to be treated.

24. The system of claim 16, wherein the ultrasound device applies low intensity volumetric ultrasound as pulse trains into subcutaneous tissue at the area to be treated and continues the application of the pulse trains until the subcutaneous tissue at the area to be treated has achieved the treatment endpoint.

25. The system of claim 16, wherein the ultrasound device adjusts a wave emitter pattern to set a depth of treatment to be that of subcutaneous tissue at the area to be treated and
applies low intensity volumetric ultrasound into the subcutaneous tissue at the area to be treated until the subcutaneous tissue at the area to be treated has achieved the treatment endpoint.

26. The system of claim 16, wherein a local anesthesia is present on a skin of the patient over the area to be treated and wherein the ultrasound device applies low intensity volumetric ultrasound through the skin where the local anesthesia is present and into the subcutaneous tissue at the area to be treated until the subcutaneous tissue at the area to be treated has achieved the treatment endpoint.

27. The system of claim 16, wherein the ultrasound device applies low intensity volumetric ultrasound with a power density level at or below 50 W/cm².

28. The system of claim 16, wherein the ultrasound device monitors a physiological parameter while applying the low intensity volumetric ultrasound and upon detecting that the physiological parameter meets a threshold, takes a remedial action.

29. The system of claim 28, wherein the remedial action comprises at least one of stopping the applying of the low intensity volumetric ultrasound and generating an alarm.

30. The system of claim 16, wherein the treatment endpoint comprises the subcutaneous tissue being treated becoming denatured.
